(19) United States
(12) Patent Application Publication
(10) Pub. No.: US 2006/0035821 A1
(43) Pub. Date: Feb. 16, 2006

(54) Cyclosporin analogs for the treatment of immunoregulatory disorders and respiratory diseases

(76) Inventors: Kevin W. Hunt, Longmont, CO (US); Laurence E. Burgess, Boulder, CO (US)

Correspondence Address: HOGAN & HARTSON LLP, ONE TABOR CENTER, 1200 17TH STREET, SUITE 1500, DENVER, CO, 80202, US

(21) Appl. No.: 10/918,917

(22) Filed: Aug. 16, 2004

(51) Int. Cl. A61K 38/13; C07K 7/64

(52) U.S. Cl. 514/11; 530/317

(57) ABSTRACT

Provided are novel cyclosporin analogs, methods for their production, and their use for treating immunoregulatory and respiratory diseases, disorders, and conditions.

### BACKGROUND OF THE INVENTION

1. Field of the Invention

This invention relates to novel cyclosporin analogs, methods for their production, and their use for treating immunoregulatory and respiratory diseases, disorders, and conditions.

2. Description of the State of the Art

Cyclosporin, originally called cyclosporine A, is the main component of a large family of cyclic undecapeptides. This family, originally isolated from cultures of Cylindrocarpon lucidum Booth and Tolypocladium Gams, is produced as secondary fungal metabolites. Cyclosporin, initially pursued for its antifungal activities, is an effective immunosuppressant, acting primarily through T-lymphocytes via inhibition of the phosphatase calcineurin. Cyclosporin reduces the production of a range of cytokines, inhibiting the activation of various cell types, including those involved in cell-mediated immunity. Due to these properties, cyclosporin remains a first line therapy in the transplantation field.

In addition to its wide use to prevent and treat organ transplant rejection, cyclosporin has been evaluated in a large range of disorders linked to immunoregulatory dysfunction and respiratory diseases. Cyclosporin, along with other calcineurin inhibitors, has been used for the treatment of nephritic syndrome, active Crohn's disease, acute ocular Behcet syndrome, endogenous uveitis, psoriasis, atopic dermatitis, rheumatoid arthritis, aplastic anemia, primary biliary cirrhosis, celiac disease and other immunoregulatory diseases. Limited evidence suggests cyclosporin is effective in patients with intractable pyoderma gangrenosum, polymyostitis/dermatomyositis or severe, corticosteroid-dependent asthma (D. Faulds, K. L. Goa, and P. Benfield; Drug Evaluation 45, 953 (1993) and P. J. Wahab, et al., Aliment Pharmacol Ther. 14, 767 (2000)). The effect of cyclosporin and other calcineurin inhibitors on inflammatory cells and their mediators make it a promising therapy for asthma, COPD (chronic obstructive pulmonary disease), idiopathic pulmonary fibrosis, and other lung diseases. Treatment of these disorders with cyclosporin is limited to patients with severe disease that are either refractory or hypersensitive to standard treatments due to adverse events including, but not limited to, hypertrichosis, gingival hyperplasia, neurological effects, gastrointestinal effects, and renal dysfunction. Chronic cyclosporin treatment requires frequent renal function monitoring due to increased incidence of kidney failure.

The mechanism of toxicity of calcineurin inhibitors such as cyclosporin has been related to the mechanism of immunosuppression (F. J. Dumont, et al., J. Exp. Med., 1992, 176:751-760). This strong link between cyclosporin mechanism of action and many cyclosporin-induced toxicities has presented a significant challenge to medicinal chemists who have tried to improve the therapeutic index of cyclosporin through chemical modification. Indeed, these efforts, to date, have failed to separate cyclosporin efficacy from its toxicity. Segregation of efficacy and toxicity of cyclosporin analogs might still be possible by altering a compound's distribution or metabolism (N. H. Signal, et. al., J. Exp. Med., 173, 619 (1991)).

The systemic toxicity of cyclosporin A therefore limits its use for the treatment of certain diseases. It is therefore desirable to find compounds for the treatment of immunoregulatory and respiratory diseases with improved systemic efficacy and safety.

### SUMMARY OF THE INVENTION

This invention provides novel cyclosporin analogs, methods to produce these compounds, and pharmaceutical compositions containing them for treating immunoregulatory and respiratory diseases, disorders, and conditions.

More particularly, the present invention provides cyclosporin analogs having the general Formula (I): 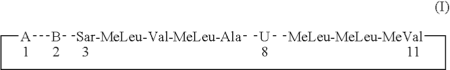
wherein residue A has the formula 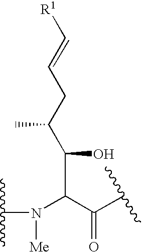
R¹ is Zₙ-cycloalkyl, Zₙ-heterocycloalkyl, Zₙ-S-cycloalkyl, Zₙ-S-heterocycloalkyl, ZₙS-ZₙR⁵, —C(═O)NR²R³, —(CH═CH₂)Ar, or —C(═O)O-alkyl wherein said alkyl is substituted with phenyl, oxo, S-heterocycle, or phenoxy, or 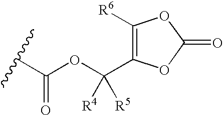
   where R⁴, R⁵ and R⁶ are independently H, C₁-C₇ alkyl, alkoxyalkyl, or alkoxycarbonyl; or R¹ is 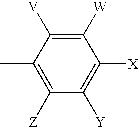
V, W, X, Y and Z are independently selected from the group consisting of H, F, Br, Cl, Zₙ-OAr, Zₙ-S-heterocycloalkyl, Zₙ-O-heterocycloalkyl, O-Zₙ-heterocycloalkyl, Zₙ-heterocycloalkyl, Zₙ-cycloalkyl, Zₙ-OAr, and Zₙ-O-alkyl, wherein at least one of V, W, X, Y or Z is other than hydrogen,
or X is 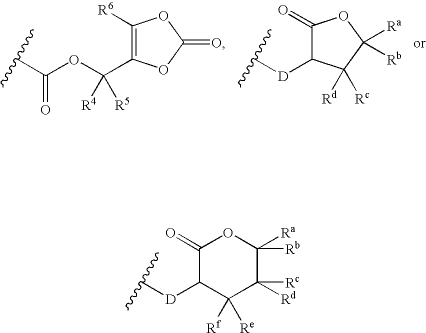
   where D is O, S or C and R^{a}, R^{b}, R^{c}, R^{d}, R^{e}, and R^{f} are independently H, F, Br, Cl, alkyl, Zₙ-O-alkyl, Zₙ-OAr,
provided that when W is F, Br, or Cl, then X is other than H, and when X is F, Br, or Cl, then W is other than H,
or X and Y together with the atoms to which they are attached form a substituted or unsubstituted heterocyclic ring;
R² and R³ are independently H, alkyl, Zₙ-Ar, and Zₙ-O-alkyl, wherein said alkyl and Ar may be substituted or unsubstituted;
Ar is substituted or unsubstituted aryl or heteroaryl;
Z is alkylene having from 1 to 4 carbons, or alkenylene or alkynylene each having from 2 to 4 carbons, wherein said alkylene, alkenylene, or alkynylene may be substituted or unsubstituted;
residue B is -αABu-, -Val-, -Thr-, or NVa-;
residue U is -(D)Ala-, (-D)Ser-, —[O-(2-hydroxyethyl)(D)Ser]-, —[O-acyl(D)Ser] or —[O-(2-acyloxyethyl)(D)Ser]-; and
n is 0, 1, 2, 3, or 4.

The compounds of the present invention have diminished plasma stability relative to known cyclosporin analogs. When administrated, the cyclosporins of the invention have potent efficacy at the site(s) of administration, while devoid of or exhibiting relatively reduced systemic activity. The cyclosporin analogs of the invention thus provide a means for the treatment of immunoregulatory and respiratory diseases, disorders, and conditions with the avoidance of unwanted systemic side effects.

In a further aspect the present invention provides a method of treating immunoregulatory and respiratory diseases, disorders, and conditions in a subject, which comprises administering to a warm-blooded animal a therapeutically effective amount of a compound of Formula (I).

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE FIGURES

The accompanying drawings, which are incorporated herein and form a part of the specification, illustrate non-limiting embodiments of the present invention, and together with the description, serve to explain the principles of the invention.

In the Figures:

FIG. 1 shows a reaction scheme for the synthesis of compound 3.

FIG. 2 shows a reaction scheme for the synthesis of compounds 16A, 16A-1, and 16B.

FIG. 3 shows a reaction scheme for the synthesis of compound 28.

FIG. 4 shows a reaction scheme for the synthesis of compounds 38 and 38A.

FIG. 5 shows a reaction scheme for the synthesis of compound 41.

FIG. 6 shows several reaction schemes for the synthesis of the cyclic carbamate derivative 44 of this invention.

FIG. 7 shows several reaction schemes for the synthesis of compound 45.

FIG. 8 shows a reaction scheme for the synthesis of compound 46.

### DETAILED DESCRIPTION OF THE INVENTION

The present invention provides cyclosporin analogs that are useful for treating immunoregulatory and respiratory diseases, disorders, and conditions. By provision of the cyclosporin analogs of the invention, which are topically active but systemically inactive, the present invention provides cyclosporin therapy to subjects for whom such therapy might otherwise be excluded, for example, due to the risk of systemic side effects.

More specifically, present invention applies the “soft drug” concept to the preparation of cyclosporin analogs. This approach limits the exposure of an active calcineurin inhibitor to organs that are sensitive (i.e., kidney) and result in toxicities while maximizing the topical exposure of an active calcineurin inhibitor to diseased tissues and organs (e.g., skin, lung, gut, eye, etc.).

A “soft drug” is a compound that is a close structural analog of a known active drug that possesses a specific metabolic liability, and provides a predictable, controlled detoxification (N. Bodor, P. Buchwald, Med. Res. Rev., 20, 58 (2000)). Most soft drugs are designed to act topically at the site of application and to be rendered inactive upon entering systemic circulation. Successful application of soft drug principles has allowed the launching of a number of drugs across several therapeutic areas. Investigations of other soft drugs continue in the area of antimicrobials, antichlolinergics, corticosteroids, β-blockers, immunoregulatory agents, analgesics, ACE inhibitors, antiarrhythmics, and others. Specifically, workers at Enanta and Norvartis have applied the soft drug concept to calcineurin inhibitors (T. Lazarova, et al., J. Med. Chem. 46, 674 (2003) and T. H. Keller, et al., in New Drugs for Asthma, Allergy and COPD; Hansel, T. T., Barnes, P. J., Eds.; Progress in Respiratory Research, Vol. 31; Karger; Basel, Switzerland 2003; pp 237-240).

Applying the soft drug principles to cyclosporin allows the segregation of its efficacy in immunoregulatory disorders (lung, skin, eye, gut, nasal, colonic, ear, oral, vaginal diseases) from its use-limiting toxicity. A “soft” analog of the cyclosporin family is highly desirable, given the current lack of safe and efficacious treatment options for immunoregulatory disorders and severe lung diseases. Accordingly, the cyclosporin analogs of this invention include “soft” analogs of all naturally occurring cyclosporins, in addition to analogs accessible by total synthesis, fermentation, enzymatic catalysis, and/or genetic engineering.

In general, one aspect of the invention provides compounds of the general Formula (I): 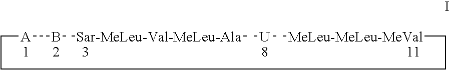
or a pro-drug or pharmaceutically acceptable salt thereof.

In Formula (I), the formula for residue A is Formula (II): 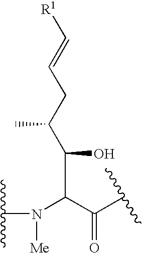
wherein
R¹ is Zₙ-cycloalkyl, Zₙ-heterocycloalkyl, Zₙ-S-cycloalkyl, Zₙ-S-heterocycloalkyl, ZₙS-ZₙR⁵, —C(═O)NR²R³, —(CH═CH₂)Ar, or —C(═O)O-alkyl, wherein said alkyl is substituted with phenyl, oxo, S-heterocycle, or phenoxy, or 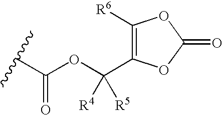
   where R⁴, R⁵ and R⁶ are independently H, C₁-C₇ alkyl, alkoxyalkyl, or alkoxycarbonyl;
   or R¹ is 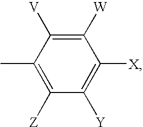
V, W, X, Y and Z are independently selected from the group consisting of H, F, Br, Cl, Zₙ-OAr, Zₙ-S-heterocycloalkyl, Zₙ-O-heterocycloalkyl, O-Zₙ-heterocycloalkyl, Zₙ-heterocycloalkyl, Zₙ-cycloalkyl, Zₙ-OAr, and Zₙ-O-alkyl,
or X is 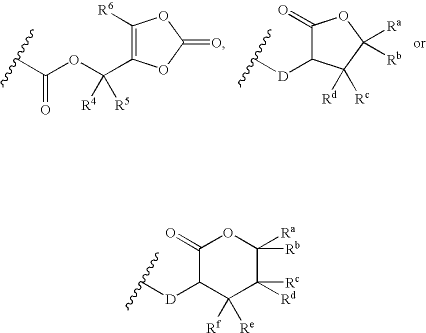
   where D is O, S or C and R^{a}, R^{b}, R^{c}, R^{d}, R^{e}, and R^{f} are independently H, F, Br, Cl, alkyl, Zₙ-O-alkyl, or Zₙ-OAr,
provided that when W is F, Br, or Cl, then X is other than H, and when X is F, Br, or Cl, then W is other than H,
or X and Y together with the atoms to which they are attached form a substituted or unsubstituted heterocyclic ring;
R² and R³ are independently H, alkyl, Zₙ-Ar, and Zₙ-O-alkyl, wherein said alkyl and Ar may be substituted or unsubstituted;
Ar is substituted or unsubstituted aryl or heteroaryl;
Z is alkylene having from 1 to 4 carbons, or alkenylene or alkynylene each having from 2 to 4 carbons, wherein said alkylene, alkenylene, or alkynylene may be substituted or unsubstituted;
residue B is -αABu-, -Val-, -Thr-, or NVa-;
residue U is -(D)Ala-, (-D)Ser-, —[O-(2-hydroxyethyl)(D)Ser]-, —[O-acyl(D)Ser] or —[O-(2-acyloxyethyl)(D)Ser]-; and
n is 0, 1, 2, 3, or 4.

In Formula (I), amino acid residues referred to by abbreviation, e.g., -Ala-, -MeLeu-, -αAbu-, etc., are, in accordance with conventional practice, to be understood as having the L-configuration unless otherwise indicated. For example, -(D)Ala- represents a residue having the D-configuration. Residue abbreviations preceded by “Me” as in the case of “MeLeu”, represent α-N-methylated residues. Individual residues of the cyclosporin molecule are numbered, as in the art, clockwise and starting with the residue, -MeBmt-corresponding to residue 1. The same numerical sequence is employed throughout the present specifications and claims.

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl, ethyl, n-propyl, isopropyl, butyl, isobutyl, sec-butyl, tert-butyl, pentyl, isopentyl, tert-pentyl, hexyl, isohexyl, and the like.

The term “cycloalkyl” refers to saturated or partially unsaturated cyclic hydrocarbon radical having from three to twelve carbon atoms, wherein the cycloalkyl may be optionally substituted independently with one or more substituents described herein. The term “cycloalkyl” further includes bicyclic and tricyclic cycloalkyl structures, wherein the bicyclic and tricyclic structures may include a saturated or partially unsaturated cycloalkyl fused to a saturated or partially unsaturated cycloalkyl or heterocycloalkyl ring or an aryl or heteroaryl ring. Examples of cycloalkyl groups include, but are not limited to, cyclopropyl, cyclobutyl, cyclopentyl, cyclohexyl, cycloheptyl, and the like.

The term “heterocycloalkyl” refers to a saturated or partially unsaturated cyclic radical of 3 to 8 ring atoms in which at least one ring atom is a heteroatom selected from nitrogen, oxygen and sulfur, the remaining ring atoms being C where one or more ring atoms may be optionally substituted independently with one or more substituent described below and wherein the heterocycloalkyl ring can be saturated or partially unsaturated. The radical may be a carbon radical or heteroatom radical. “Heterocycloalkyl” also includes radicals where heterocycle radicals are fused with aromatic or heteroaromatic rings, such as 2-coumaranone and phthalide rings. Examples of heterocycloalkyl rings include, but are not limited to, lactones, pyrrolidine, piperidine, piperazine, tetrahydropyranyl, morpholine, thiomorpholine, homopiperazine, phthalimide, and derivatives thereof.

“Aryl” means a monovalent aromatic hydrocarbon monocyclic radical of 6 to 10 ring atoms or a polycyclic aromatic hydrocarbon, optionally substituted independently with one or more substituents described herein. More specifically the term aryl includes, but is not limited to, phenyl, 1-naphthyl, 2-naphthyl, and derivatives thereof.

“Heteroaryl” means a monovalent monocyclic aromatic radical of 5 to 10 ring atoms or a polycyclic aromatic radical, containing one or more ring heteroatoms selected from N, O, or S, the remaining ring atoms being carbon. The aromatic radical is optionally substituted independently with one or more substituents described herein. Examples include, but are not limited to, furyl, thienyl, pyrrolyl, pyridyl, pyrazolyl, pyrimidinyl, imidazolyl, pyrazinyl, indolyl, thiophen-2-yl, quinolyl, benzopyranyl, thiazolyl, and derivatives thereof.

In general, the various moieties or functional groups of the compounds of Formula (I) may be optionally substituted by one or more substituents. Examples of substituents suitable for purposes of this invention include, but are not limited to, halo, oxo, alkyl, allyl, alkenyl, alkynyl, heteroalkyl, heteroallyl, heteroalkenyl, heteroalkynyl, alkoxy, heteroalkoxy, cycloalkyl, heterocycloalkyl, —OR, —NO₂, —CN, —CO₂R, —(C═O)R, —O(C═O)R, —O-alkyl, —OAr, —SH, —SR, —SOR, —SO₂R, —S—Ar, —SOAr, —SO₂Ar, Ar, —(C═O)NR²R³, NR²R³, —NR²R³, —PO₃H₂, —SO₃H₂, where Ar is aryl or heteroaryl, and wherein said alkyl, allyl, alkenyl, alkynyl, heteroalkyl, heteroallyl, heteroalkenyl, heteroalkynyl, alkoxy, heteroalkoxy, cycloalkyl, heterocycloalkyl, Ar, R¹, R², and R³ may be further substituted or unsubstituted.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. Accordingly, this invention also includes racemates and resolved enantiomers, and diastereomers compounds of the Formula (I). The methods for the determination of stereochemistry and the separation of stereoisomers are well known in the art (see discussion in Chapter 4 of “Advanced Organic Chemistry”, 4th edition J. March, John Wiley and Sons, New York, 1992).

In addition to compounds of the Formula (I), the invention also includes solvates, pharmaceutically acceptable prodrugs, pharmaceutically active metabolites, and pharmaceutically acceptable salts of such compounds.

The term “prodrug” means compounds that are rapidly transformed in vivo to yield the parent compound of the formulas of this invention, for example by hydrolysis in blood. Functional groups which may be rapidly transformed, by metabolic cleavage, in vivo form a class of groups reactive with the carboxyl group of the compounds of this invention. They include, but are not limited to groups such as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group.

Prodrugs of a compound may be identified using routine techniques known in the art. Various forms of prodrugs are known in the art. For examples of such prodrug derivatives, see, for example, a) Design of Prodrugs, edited by H. Bundgaard, (Elsevier, 1985) and Methods in Enzymology, Vol. 42, pp. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs”, by H. Bundgaard p. 113-191 (1991); c) H. Bundgaard, Advanced Drug Delivery Reviews, 8, 1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

A “pharmaceutically acceptable salt” is a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable sale. Examples of pharmaceutically acceptable salts include those salts prepared by reaction of the compounds of the present invention with a mineral or organic acid or an inorganic base, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitromenzoates, hydroxybenzoates, methoxybenzoates, phthalates, sulfonates, xylenesulfonates, pheylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

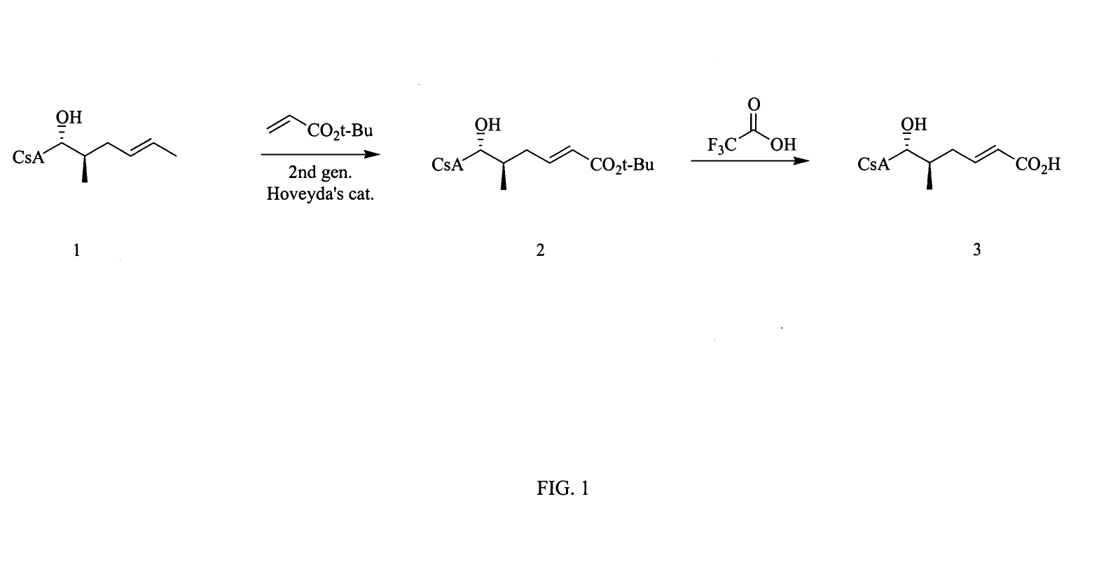

The inventive compounds may be prepared using the reaction routes and synthesis schemes as described below, employing the techniques available in the art using starting materials that are readily available. FIGS. 1-4 show examples of the synthesis of specific compounds having the general Formula (I). The starting material for the reactions shown in FIGS. 1-4 may be, for example, but not limited to, a fermentation product or a synthetic product made by solution phase chemistry. The starting material as a fermentation product may be made from highly productive strains, including but not limited to, a Sesquicillopsis rosariensis G. ARNOLD F605; Tolypocladium inflatum wb6-5; Fusant, Tolypocladium inflatum KD461 (see, for example, U.S. Pat. Nos. 5,256,547 and 5,856,141). Alternatively, the starting material may be made by solution phase chemistry either by sequentially assembling amino acids or by linking suitable small peptide fragments, where the units are linked by, for example, amide, ester or hydroxylamine linkages (see, for example, Muller, Methoden der organischen, Chemie vol. XV/2, pp 1-364, Thieme Verlag, Stuttgart, 1974; Stewart, Young, Solid Phase Peptide Synthesis, pp 31 to 34, 71 to 82, Pierce Chemical Company, Rockford, 1984; Bodanszky, Kausner, Ondetti, Peptide Synthesis, pp 85 to 128, John Wiley & Sons, New York, 1976).

The process for the preparation of compounds of Formula (I) comprises reacting a compound cyclosporin A with an olefin having a terminal double bond using a catalyst such as Hoveyda's 2^{nd} generation catalyst ruthenium catalyst (Org. Biomol. Chem., 2004, 2:8-23), or any other suitable catalyst, such as Grubb's ruthenium alkylidine, Grubbs dihydroimidazole ruthenium, Shrock-Hoveyda molybdenum catalysts or benzylidine catalysts [(see (a) U.S. Pat. No. 6,111,121; (b) Reviews: Synlett, 1999, 2:267; (c) Reviews: Ivin, K. J., Mol, J. C., Olefin Metathesis and Metathesis Polymerization, 2^{nd} ed., Academic Press, New York, 1997; (d) J. Org. Chem., 2000, 65, 2204-2207] or molybdenum catalysts [see (a) J. Am. Chem. Soc., 1990, 112:3875; (b) J. Am. Chem. Soc., 1996, 118, 10926-10927] in the presence of a lithium salt such as lithium bromide, lithium chloride, lithium trifluoroacetate, lithium triflate of a Lewis acid such as titanium isopropoxide in an organic solvent.

After the metathesis reaction, the reaction products can be further reacted to produce the compounds of the present invention. For example, FIG. 1 shows the reaction scheme for the synthesis of compound 2, obtained by the reaction between cyclosporin A and t-butyl acrylate catalyzed by Hoveyda's 2^{nd} generation catalyst. Compound 2 is then treated with trifluoroacetic acid to provide compound 3.

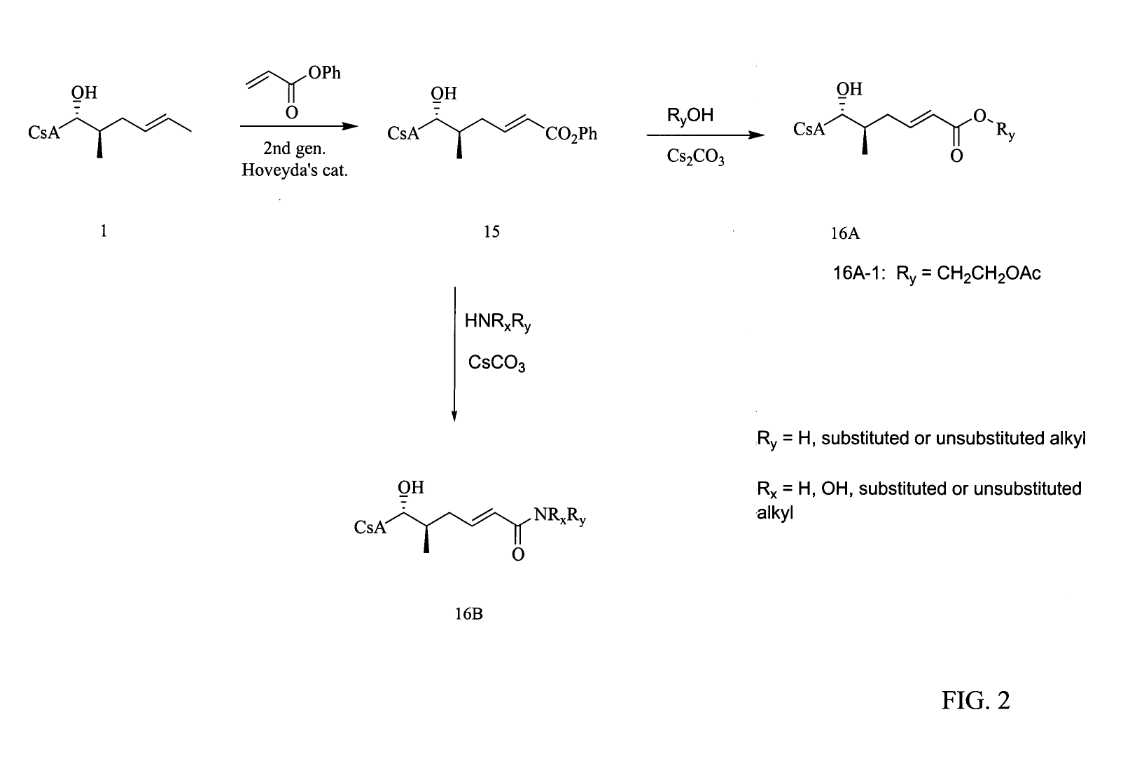

FIG. 2 shows the reaction scheme for the synthesis of compounds having the general formula 16A or 16B, obtained by the reaction of cyclosporin A and phenyl acrylate catalyzed by Hoveyda's 2^{nd} generation catalyst to provide compound 15. Compound 15 is then converted to compound 16A or 16B by treating compound 15 with a compound having the formula R_{y}OH or HNRₓR_{y} and a base such as cesium carbonate, where Rₓ is H, OH or a substituted or unsubstituted alkyl R_{y} is H or a substituted or unsubstituted alkyl. The success of this reaction is based on the recognition by the inventors that compound 15 is a more reactive ester due to the phenyl group. This phenyl ester not only allows for a facile transesterification with improved yield and efficiency, but further allows for the easy conversion of the phenyl ester to an amide or hydroxamate without affecting the alkyl ester that is introduced as a result of the conversion (e.g., the acetate group of compound 16).

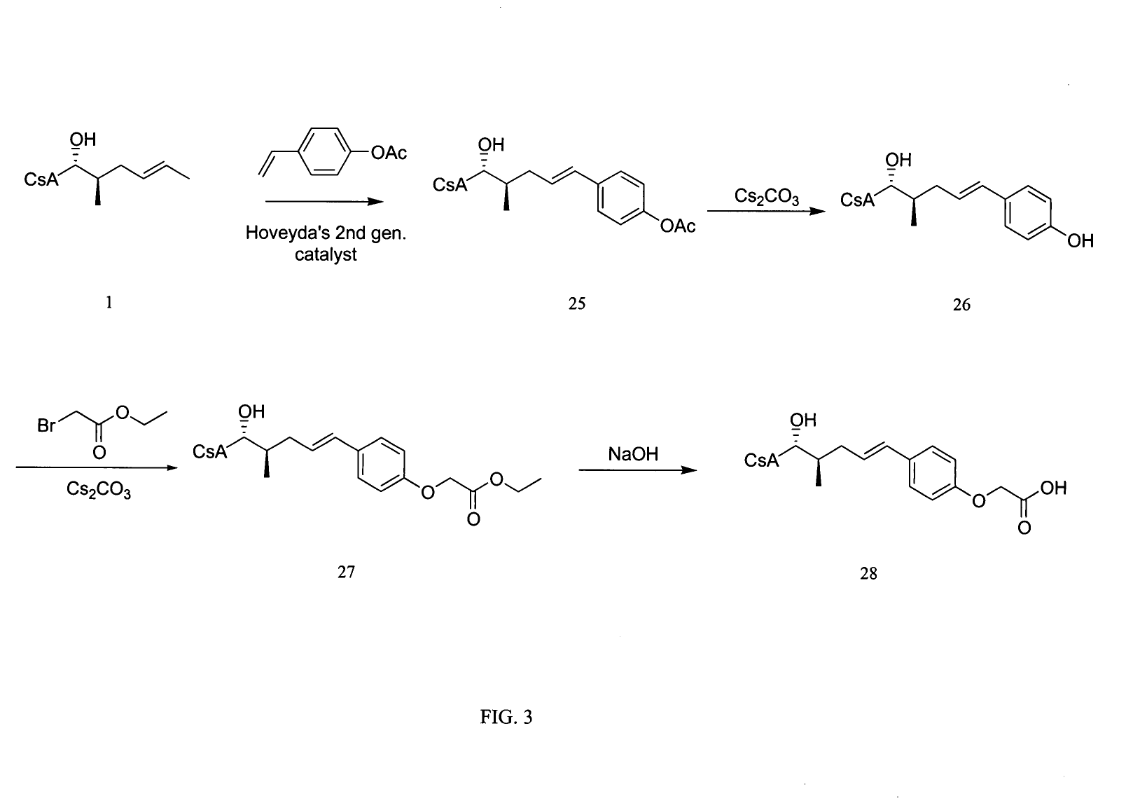

FIG. 3 shows the reaction scheme for he synthesis of compound 28, the synthesis of which is described in Example 7.

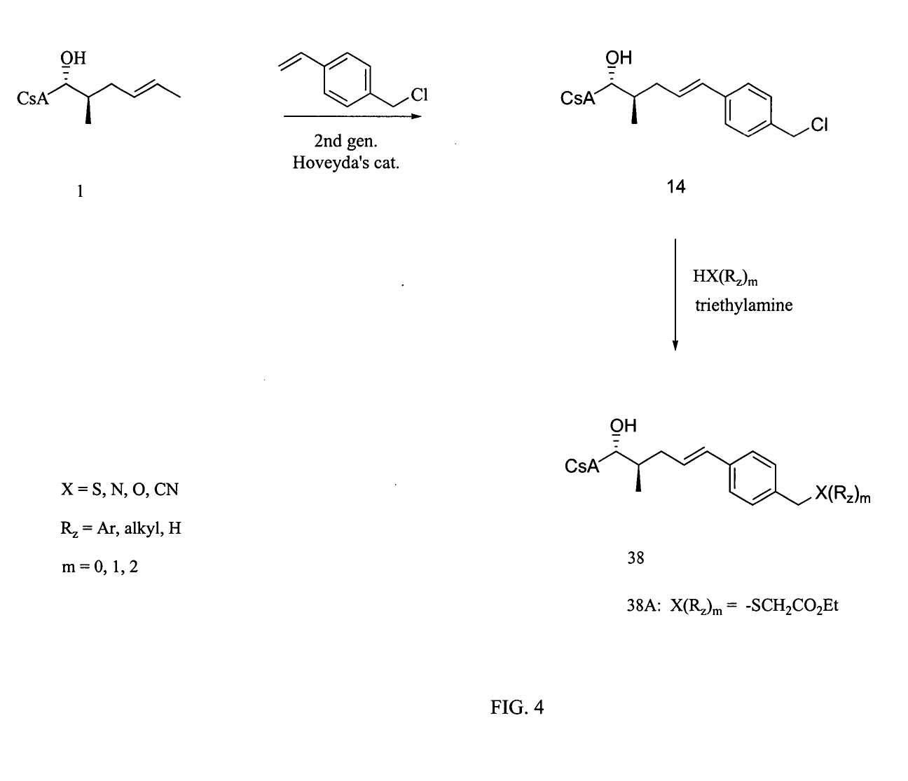

FIG. 4 shows the reaction scheme for the synthesis of compound 38, the synthesis of which is described in Example 8. The preparation of compound 38 utilizes the unique intermediate 14, which is a benzyl chloride that is suitably electrophilic and allows for the synthesis of a variety of compounds by reacting compound 14 with a nucleophile. Examples of suitable nucleophiles include, but are not limited to, aryl thiols, alkyl thiols, substituted alkyl thiols, amines, anilines, alcohols, phenols, and cyanide.

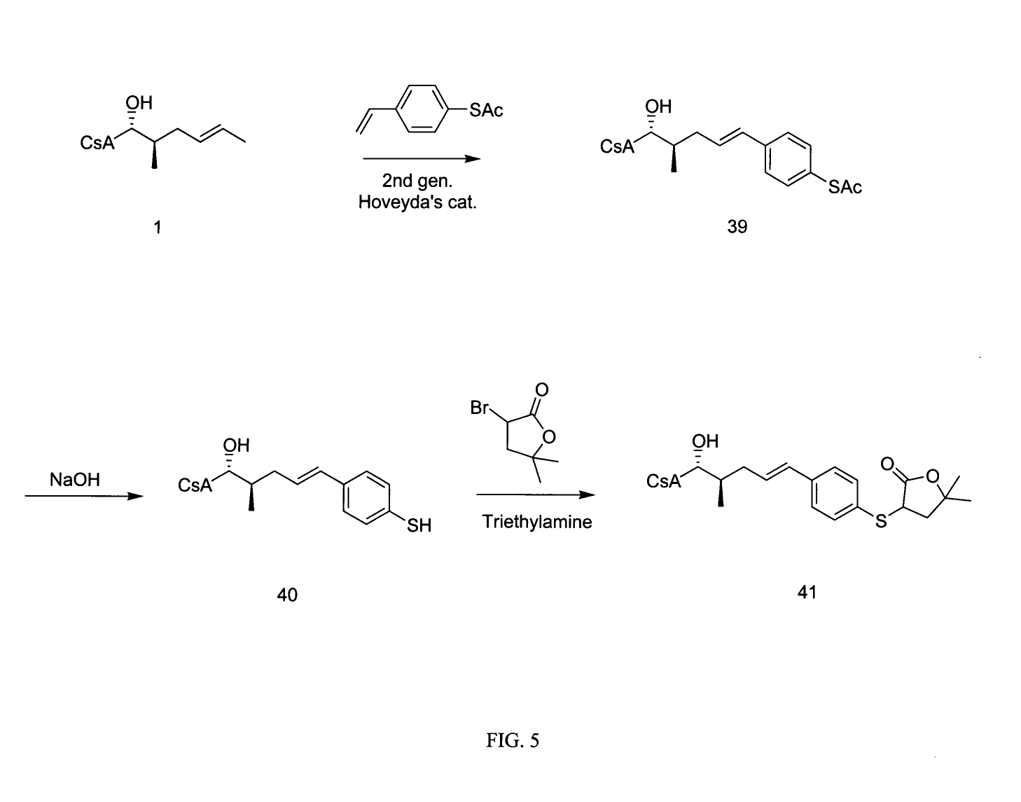

FIG. 5 shows the reaction scheme for the synthesis of compound 41, the synthesis of which is described in Example 33.

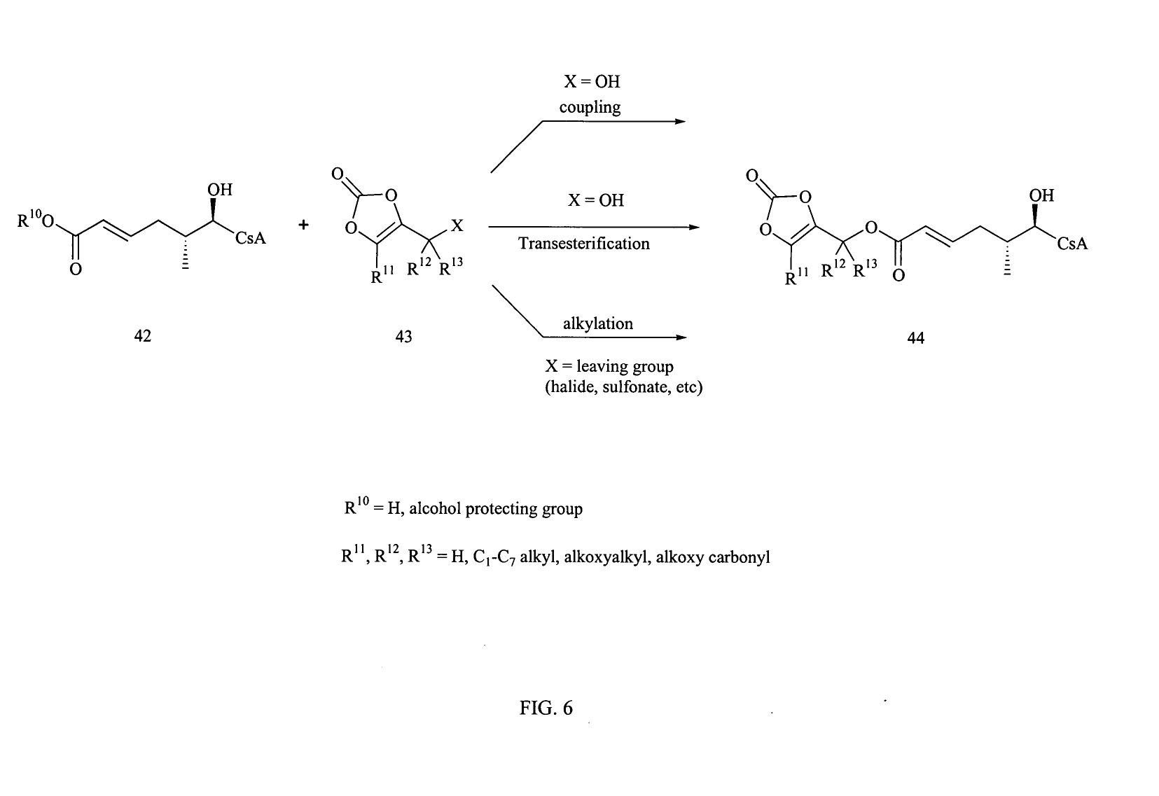

FIG. 6 shows several reaction schemes for the synthesis of cyclic carbamate derivatives 44 of this invention. The cyclic carbamate can be prepared, for example, from the acid 43 and the alcohol 42 using standard coupling procedures, e.g., by alkylation of the acid 43 with an appropriate electrophile, or by transesterification.

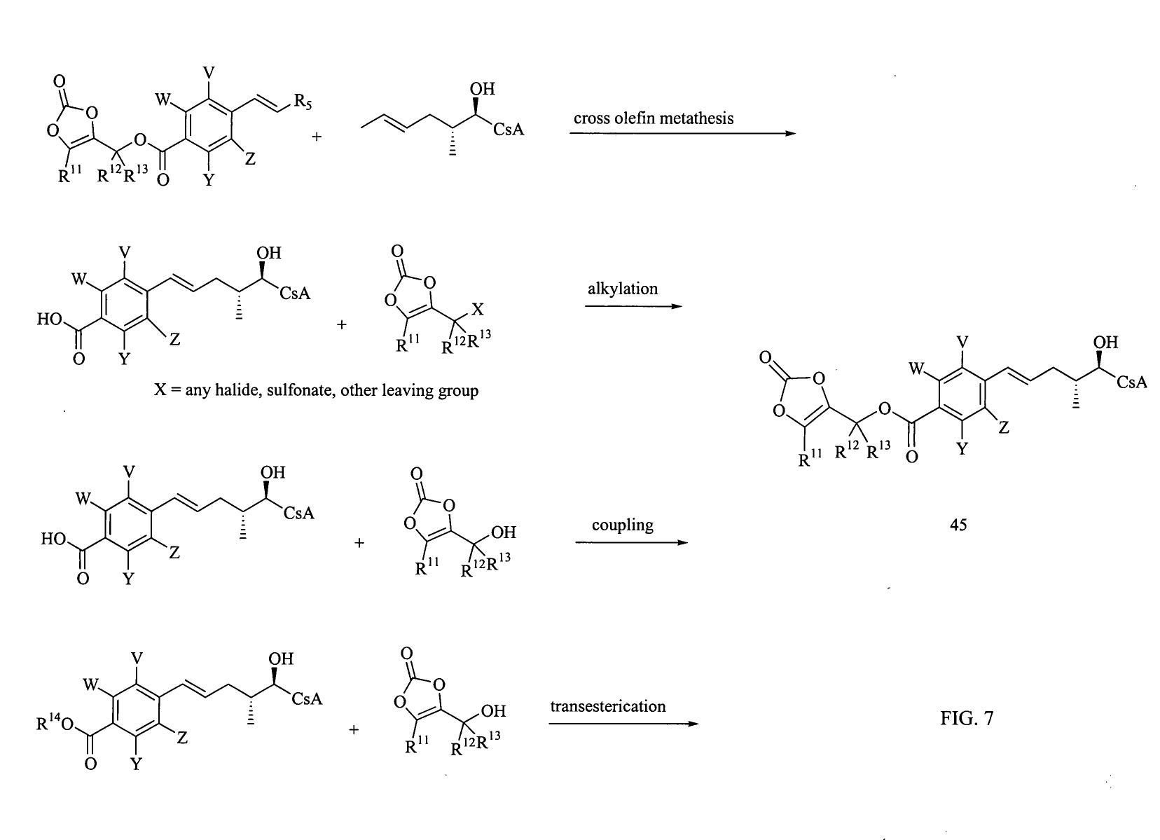

FIG. 7 shows several reaction schemes for the synthesis of derivatives of this invention having the general formula 45. For example, the derivatives shown in FIG. 7 can be prepared from cyclosporin A and an appropriate olefin by cross olefin metathesis, by alkylation of the acid with the appropriate electrophile, by coupling of the acid to the corresponding alcohol, or by transesterification.

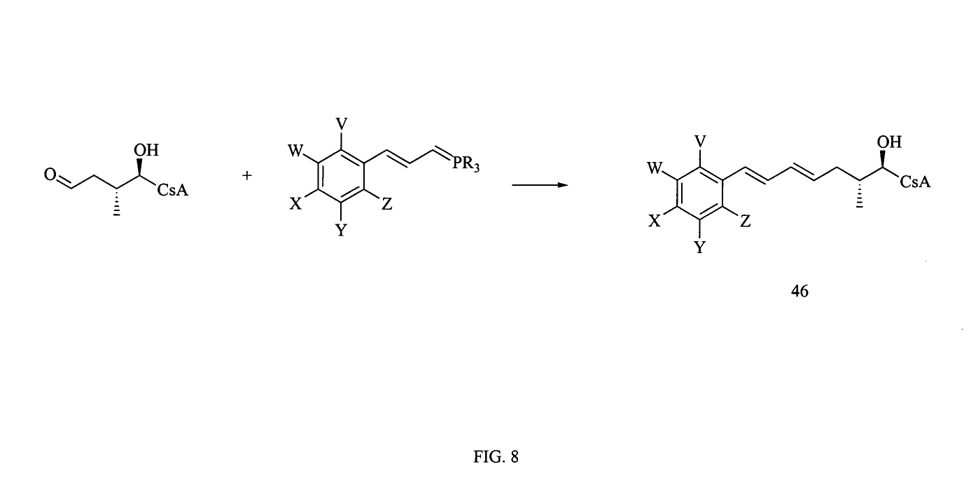

FIG. 8 shows the reaction scheme for the synthesis of derivatives of this invention having the general formula 46. This derivative can be prepared, for example, by a Wittig type reaction with the appropriate aldehyde.

The cyclosporins of the present invention are useful for the treatment of diseases or conditions responsive to or requiring anti-inflammatory, immunosuppressive, or related therapy, e.g. for topical administration for the treatment of such diseases or conditions of the eye, nasal passages, buccal cavity, colon, skin, intestinal tract, airway, or lung. In particular, the cyclosporins of the present invention permit topical anti-inflammatory, immunosuppressive or related therapy with the concomitant avoidance or reduction of undesirable systemic side effects, for example general systemic immunosuppression.

Cyclosporins of the invention are particularly useful for the treatment of diseases and conditions of the airways or lung, in particular inflammatory or obstructive airways disease. They are especially useful for the treatment of diseases or conditions of the airways or lung associated with or characterized by inflammatory cell infiltration or other inflammatory event accompanied by the accumulation of inflammatory cells, e.g., eosinophils and/or neutrophils.

The cyclosporins of the invention are particularly useful for the treatment of asthma of whatever type of genesis, including both intrinsic and, especially, extrinsic asthma. For example, they are useful for the treatment of atopic and non-atopic asthma, including allergic asthma, bronchitic asthma, exercise-induced asthma, occupational asthma, asthma induced following bacterial infection and other non-allergic asthmas. Treatment of asthma is also to be understood as embracing treatment of “wheezy-infant syndrome,” that is treatment of subjects, e.g., of less than 4 to 5 years of age, exhibiting wheezing symptoms, in particular at night, and diagnosed or diagnosable as “wheezy infants,” an established patient category of major medical concern and now more correctly identified as incipient or early-phase asthmatics. Cyclosporins of the invention are in particular useful for the treatment of asthma in subjects whose asthmatic status is either steroid-dependent or steroid-resistant.

Cyclosporins of the invention are also useful for the treatment of bronchitis or for the treatment of chronic or acute airways obstruction associated therewith. Cyclosporins of the invention may be used for the treatment of bronchitis of whatever type or genesis, including, for example, acute bronchitis, arachidic bronchitis, catarrhal bronchitis, chronic bronchitis, croupous bronchitis, phthinoid bronchitis and so forth.

Cyclosporins of the invention are in addition useful for the treatment of pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, berylliosis, chalicosis, ptilosis, siderosis, silicosis, tabacosis and, in particular, byssinosis.

Cyclosporins of the invention may also be used for the treatment of eosinophil-related disorders of the airways (e.g., involving morbid eosinophilic infiltration of pulmonary tissues) including hypereosinophilia as it affects the airways and/or lungs as well as, for example, eosinophil-related disorders of the airways consequential or concomitant to Loffler's Syndrome, eosinophilic pneumonia, parasitic (in particular metazoan) infestation (including tropical eosinophilia), bronchopulmonary aspergillosis, polyarteritis nodosa (including Churg-Strauss Syndrome), eosinophilic granuloma and eosinophil-related disorders affecting the airways occasioned by drug reaction.

Cyclosporins of the invention may also be used to treat any disease or condition of the airways or lung requiring immunosuppressive therapy, e.g., for the treatment of autoimmune diseases of, or as they affect, the lungs (for example, for the treatment of sarcoidosis, alveolitis or chronic hypersensitivity pneumonitis) or for the maintenance of allogenic lung transplant, e.g., following lung or heart lung transplantation.

The amount of a given agent that will correspond to such an amount will vary depending upon factors such as the particular compound, disease condition and its severity, and the identity (e.g., weight) of the mammal in need of treatment, but can nevertheless be routinely determined by one skilled in the art. The term “treatment” is intended to include at least the mitigation of a disease condition in a mammal, such as a human, and includes, but is not limited to, preventing the disease condition from occurring in a mammal, particularly when the mammal is found to be predisposed to having the disease condition but has not yet been diagnosed as having it; modulating and/or inhibiting the disease condition; and/or alleviating the disease condition.

When used in relation to the treatment of diseases of the airways and lungs, in particular asthma, the term “treatment” is to be understood as embracing both symptomatic and prophylactic modes, that is the immediate treatment, for example, of acute inflammation (symptomatic treatment) as well as advance treatment to prevent, ameliorate or restrict long term symptomatology (prophylactic treatment). For example, in the case of asthma, the present invention includes symptomatic treatment to ameliorate acute inflammatory events as well as prophylactic treatment to inhibit on-going inflammatory status and to ameliorate future bronchial exacerbation associated therewith.

The present invention further relates to a method of preventing or treating an inflammatory or autoimmune disorder in a subject while eliminating or reducing the toxicity associated with the administration of cyclosporin A, through the systemic administration of a therapeutically effective amount of a pharmaceutical composition comprising at least one cyclosporin analog of the following Formula (I) or a pro-drug or pharmaceutically acceptable salt thereof. Inflammatory or immune disorders that can be treated by the cyclosporins of the present invention include, but are not limited to, rheumatoid arthritis, inflammatory bowel disease, psoriasis, atopic dermatitis, asthma, allergic rhinitis, and chronic obstructive pulmonary disease.

The present invention also provides methods of prevention of organ transplantation rejection in a subject by administering to the subject therapeutically effective amounts of one or more of the cyclosporin analogs of the present invention with or without the concurrent use of other known treatments.

As immunosuppressants, the compounds of Formula (I) are useful when administered for the prevention of immune-mediated tissue or organ graft rejection. Examples of transplanted tissues and organs which suffer from these effects are heart, kidney, liver, medulla ossium, skin, cornea, lung, pancreas, intestinum tenue, limb, muscle, nervus, duodenum, small-bowel, pancreatic-islet-cell, and the like; as well as graft-versus-host diseases brought about by medulla ossium transplantation. The regulation of the immune response by the compounds of the invention would also find utility in the treatment of autoimmune diseases, such as rheumatoid arthritis, systemic lupus erythematosis, hyperimmunoglobulin E, Hashimoto's thyroiditis, multiple sclerosis, progressive systemic sclerosis, myasthenia gravis, type I diabetes, uveitis, allergic encephalomyelitis, glomerulonephritis, and the like; and further infectious diseases caused by pathogenic microorganisms, such as HIV. In the particular cases of HIV-1, HIV-2 and related retroviral strains, inhibition of T-cell mitosis would suppress the replication of the virus, since the virus relies upon the host T-cell's proliferative functions to replicate.

Cyclosporins of the invention may be administered by routes including, but not limited to, the pulmonary route (inhalation), nasal administration, rectal administration (e.g. suppository or enema form), dermally (topically to the skin), or orally. When administrated, the cyclosporins of the invention will have potent efficacy at the site(s) of administration, while devoid of, or exhibit relatively reduced, systemic activity. ts.

For example, certain cyclosporins of the invention preferably will be administered topically within the airways, e.g. by the pulmonary route, by inhalation. While having potent efficacy when administered topically, cyclosporins of the invention are devoid of, or exhibit relatively reduced, systemic activity, e.g. following oral administration. Cyclosporins of the invention thus provide a means for the treatment of diseases and conditions of the airways or lung with the avoidance of unwanted systemic side effect, e.g., consequent to inadvertent swallowing of drug substance during inhalation therapy.

Cyclosporins of the invention can also be administered dermally, i.e. topically to the skin, for example for the treatment of cutaneous diseases mediated by immune mechanisms, e.g., psoriasis, contact dermatitis, atopic dermatitis, alopecia greata, erythema multiforma, dermatisis herpetiformis, scleroderma, vitiligo, hypersensitivity angiitis, urticaria, bullous pemphigoid, lumpus erythematosus, pemphisus, epidermolysis bullosa acquisita, and other inflammatory or allergic conditions of the skin. Optionally, the cyclosporins of the invention are co-administered together with anti-inflammatory, immunosuppressive, or other pharmacologically active agents, e.g., corticosteroids, antihistamines, antibiotics, antifungals, etc.

In one aspect of this invention, the compounds of this invention or the pharmaceutical salts or prodrugs thereof may be formulated into pharmaceutical compositions for administration to animals or humans to treat or prevent an immunoregulatory or respiratory disease, disorder, or condition. In order to use a compound of the Formula (I), it is normally formulated in accordance with standard pharmaceutical practice as a pharmaceutical composition. According to this aspect of the invention there is provided a pharmaceutical composition that comprises a compound of the Formula (I), or a pharmaceutically acceptable salt or in vivo cleavable prodrug thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose. methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient that is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

Use of controlled-release oral dosage forms that comprise a tablet or capsule containing a plurality of particles of a cyclosporin of this invention dispersed in a swellable/erodible polymer may be used. Further, controlled release oral dosage forms of the cyclosporins of the invention may be used for continuous, sustained administration to the upper gastrointestinal tract of a patient. The majority of the dose of cyclosporins of the invention may be delivered, on an extended release basis, to the stomach, duodenum, and upper regions of the small intestine, with delivery of the drug to the lower gastrointestinal tract and colon substantially restricted. A variety of technologies, including hydrophilic, water-swellable, crosslinked, polymers that maintain physical integrity over the dosage lifetime but thereafter rapidly dissolve may be utilized for delivery of the cyclosporins of the invention.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of a compound of this invention that is combined with one or more excipients to produce a single dosage form will necessarily vary depending upon the host treated and the particular route of administration. For example, a formulation intended for oral administration to humans will may contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

The size of the dose for therapeutic or prophylactic purposes of a compound of Formula (I) will naturally vary according to the nature and severity of the conditions, the age and sex of the animal or patient and the route of administration, according to well known principles of medicine.

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other cyclosporin analogs of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

### EXAMPLES

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated. Tetrahydrofuran (THF), N,N-dimethylformamide (DMF), dichloromethane (DCM), toluene, dioxane and 1,2-difluoroethane were purchased from Aldrich in Sure seal bottles and used as received. Hoveyda's 2^{nd} generation catalyst was purchased from Aldrich.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

### Example 1

### Procedure A: Synthesis of Compound 3

The reaction scheme for the synthesis of compound 3 according to procedure A is shown in FIG. 1.

Step 1: Synthesis of compound 2: To a solution of cyclosporin A (1.61 g, 1.34 mmol) in dichloromethane (3.4 mL) under N₂ atmosphere was added t-butyl acrylate (2.57 g, 20.1 mmol) and Hoveyda's 2^{nd} generation catalyst (84 mg, 0.13 mmol). The resulting green solution was heated to reflux under nitrogen for 16 hours. The reaction mixture was chromatographed on silica eluting with a gradient of dichloromethane, dichloromethane/MeOH (40:1), dichloromethane/MeOH (20:1), to afford 1.60 g of compound 2 as a gray solid (93% yield). MS (APCI+) m/z 1288 (M+1) detected.

Step 2: Synthesis of compound 3: A solution of compound 2 (0.054 g, 0.042 mmol) in dichloromethane/TFA (4 mL, 1:1) was stirred at room temperature for 2 hours. The mixture was concentrated under reduced pressure and chromatographed on silica eluting with 10% acetonitrile in ethyl acetate with 0.25% acetic acid. The desired compound 3 was obtained in 48% yield. MS (APCI−) m/z 1231 (M−1) detected.

### Example 2

### Synthesis of Compound 4

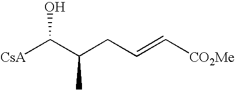

Prepared according to Procedure A, Step 1 from cyclosporin A and methyl maleate. The crude product was chromatographed on silica eluting with a gradient of dichloromethane, 2.5% MeOH in dichloromethane, 5% MeOH in dichloromethane to afford compound 4 as a pale gray solid (88% yield). MS (APCI+) m/z 1246 (M+1) detected.

### Example 3

### Synthesis of Compound 5

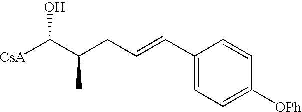

Prepared according to Procedure A, Step 1 from cyclosporin A and 4-phenoxystyrene. The crude product was chromatographed on silica eluting with a gradient of dichloromethane, dichloromethane/MeOH (40:1) and dichloromethane/MeOH (20:1) to afford compound 5 in 98% yield. MS (APCI+) m/z 1357 (M+1) detected.

### Example 4

### Synthesis of Compound 6

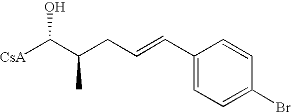

Prepared according to Procedure A, Step 1 from cyclosporin A and 4-bromostyrene. The crude product was chromatographed on silica eluting with a gradient of dichloromethane, dichloromethane/MeOH (97.5:2.5) and dichloromethane/MeOH (95:5) to afford compound 6 in 94% yield. MS (APCI+) m/z 1342, 1344 (M+1; Br pattern) detected.

### Example 5

### Synthesis of Compound 7

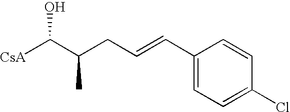

Prepared according to Procedure A, Step 1 from cyclosporin A and 4-chlorostyrene. The crude product was chromatographed on silica eluting with a gradient of dichloromethane, dichloromethane/MeOH (40:1) and dichloromethane/MeOH (20:1) to afford compound 7 in 94% yield. MS (APCI+) m/z 1298, 1300 (M+1; Cl pattern) detected.

### Example 6

### Synthesis of Compound 8

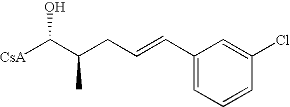

Prepared according to Procedure A, Step 1 from cyclosporin A and 3-chlorostyrene. The crude product was chromatographed on silica eluting with a gradient of dichloromethane, dichloromethane/MeOH (40:1) and dichloromethane/MeOH (20:1) to afford compound 8 in 97% yield. MS (APCI+) m/z 1298, 1300 (M+1; Cl pattern) detected.

### Example 7

### Synthesis of Compound 9

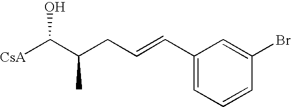

Prepared according to Procedure A, Step 1 from cyclosporin A and 3-bromostyrene. The crude product was chromatographed on silica eluting with a gradient of dichloromethane, dichloromethane/MeOH (98:2), dichloromethane/MeOH (96.5:3.5) and dichloromethane/MeOH (95:5) to afford compound 9 in 91% yield. MS (ESI+) m/z 1342, 1344 (M+1; Br pattern) detected.

### Example 8

### Synthesis of Compound 10

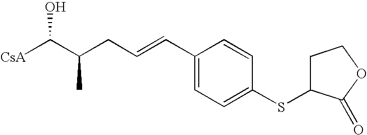

Prepared according to Procedure A, Step 1 from cyclosporin A and 3-(4-vinylphenylsulfanyl)-dihydrofuran-2-one. The crude product was chromatographed on silica eluting with a gradient of dichloromethane, dichloromethane/MeOH (97.5:2.5) and dichloromethane/MeOH (95:5). The residue was then purified by reverse phase HPLC to afford compound 10 in 67% yield. MS (APCI+) m/z 1380 (M+1) detected.

### Example 9

### Synthesis of Compound 11

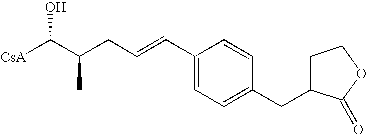

Prepared according to Procedure A, Step 1 from cyclosporin A and 3-(4-vinylbenzyl)-dihydrofuran-2-one. The crude product was chromatographed on silica eluting with a gradient of 2-6% MeOH in dichloromethane. The residue was then purified by reverse phase HPLC to afford compound 11 in 41% yield. MS (APCI−) m/z 1361 (M−1) detected.

### Example 10

### Synthesis of Compound 12

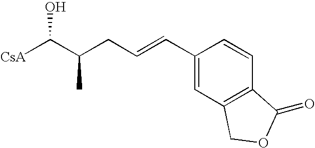

Prepared according to Procedure A, Step 1 from cyclosporin A and 5-vinyl-3H-isobenzofuran-1-one. The crude product was chromatographed on silica eluting with a gradient of dichloromethane, dichloromethane/MeOH (97.5:2.5) and dichloromethane/MeOH (95:5). The residue was then purified by reverse phase HPLC to afford compound 12 in 36% yield. MS (APCI+) m/z 1320 (M+1) detected.

### Example 11

### Synthesis of Compound 13

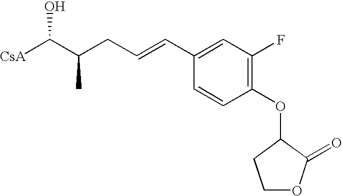

Prepared according to Procedure A, Step 1 from cyclosporin A and 3-(2-fluoro-4-vinylphenoxy)-dihydrofuran-2-one. The crude product was chromatographed on silica eluting with a gradient of 2-6% MeOH in dichloromethane. The residue was then purified by reverse phase HPLC to afford compound 13 in 73% yield. MS (APCI−) m/z 1380 (M−1) detected.

### Example 12

### Synthesis of Compound 14

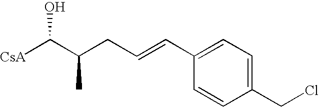

Prepared according to Procedure A, Step 1 from cyclosporin A and 4-chlorostyrene. The crude product was chromatographed on silica eluting with a gradient of 2-4% MeOH in dichloromethane. The residue was then purified by reverse phase HPLC to afford compound 14 in 34% yield. MS (APCI−) m/z 1314.6 (M+1) detected.

### Example 13

### Procedure B: Synthesis of Compound 16A-1

The reaction scheme for the synthesis of compound 16A-1 according to procedure B is shown in FIG. 2.

Step 1: Synthesis of compound 15: Prepared according to Procedure A, Step 1 from cyclosporin A and phenyl acrylate. The crude product was chromatographed on silica eluting with a gradient of dichloromethane, dichloromethane/MeOH (40:1), dichloromethane/MeOH (20:1), to afford compound 15 as a gray solid (95% yield). MS (APCI+) m/z 1308 (M+1) detected.

Step 2: Synthesis of compound 16A-1: A solution of compound 15 (0.043 g, 0.033 mmol) and ethylene glycol monoacetate (technical grade containing 25% ethylene glycol and 25% ethylene glycol diacetate, 0.068 g, 0.66 mmol) in dioxane (0.30 mL) was treated with Cs₂CO₃ (0.015 g, 0.046 mmol). The reaction vial was capped and heated to 50° C. for 1 hour. The cooled solution was chromatographed on silica packed with ethyl acetate/hexanes (1:1), eluting with a gradient of ethyl acetate/hexanes (1:1), ethyl acetate/hexanes (7:3), ethyl acetate, 3% MeOH in ethyl acetate. Compound 16A-1 was obtained as white solid (22 mg, 51% yield). MS (APCI+) m/z 1318 (M+1) detected.

### Example 14

### Synthesis of Compound 17

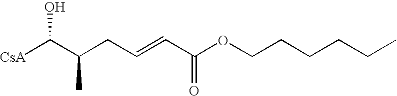

Prepared according to Procedure B, Step 2 from compound 15 (Example 13) and hexan-1-ol. The crude product was chromatographed on silica packed with ethyl acetate/hexanes (1:1), eluting with a gradient of ethyl acetate/hexanes (1:1), ethyl acetate, and 4% MeOH in ethyl acetate to provide compound 17 in 90% yield. MS (APCI+) m/z 1316 (M+1) detected.

### Example 15

### Synthesis of Compound 18

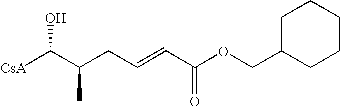

Prepared according to Procedure B, Step 2 from compound 15 (Example 13) and cyclohexylmethanol. The crude mixture was partitioned between 1N NaOH and chloroform and the organic layer was dried, filtered and concentrated under reduced pressure. The residue was chromatographed on silica packed with ethyl acetate/hexanes (1:1), eluting with a gradient of ethyl acetate/hexanes (1:1), ethyl acetate, and 4% MeOH in ethyl acetate to provide compound 18 in 91% yield. MS (APCI+) m/z 1328 (M+1) detected.

### Example 16

### Synthesis of Compound 19

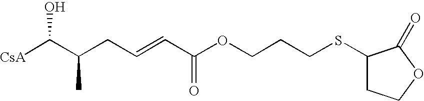

Prepared according to Procedure B, Step 2 from compound 15 (Example 13) and 3-(3-hydroxypropylsulfanyl)-dihydrofuran-2-one. The reaction mixture was heated to 70° C. for 30 hours. The crude product was chromatographed on silica packed with ethyl acetate/hexanes (1:1), eluting with a gradient of ethyl acetate/hexanes (1:1), ethyl acetate, and 4% MeOH in ethyl acetate to provide compound 19 in 49% yield. MS (APCI+) m/z 1390 (M+1) detected.

### Example 17

### Synthesis of Compound 20

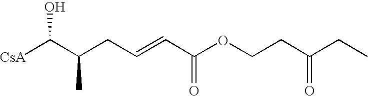

Prepared according to Procedure B, Step 2 from compound 15 (Example 13) and 6-hydroxyhexan-3-one. The crude product was chromatographed on silica packed with ethyl acetate/hexanes (1:1), eluting with a gradient of ethyl acetate/hexanes (1:1), ethyl acetate, and 4% MeOH in ethyl acetate to provide compound 20 in 72% yield. MS (APCI+) m/z 1316 (M+1) detected.

### Example 18

### Synthesis of Compound 21

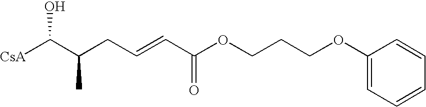

Prepared according to Procedure B, Step 2 from compound 15 (Example 13) and 3-phenoxypropan-1-ol. The crude product was chromatographed on silica packed with ethyl acetate/hexanes (1:1), eluting with a gradient of ethyl acetate/hexanes (1:1), ethyl acetate, and 4% MeOH in ethyl acetate to provide compound 21 in 90% yield. MS (APCI+) m/z 1366 (M+1) detected.

### Example 19

### Synthesis of Compound 22

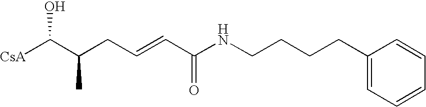

Prepared according to Procedure B, Step 2 from compound 15 (Example 13) and 4-phenylbutylamine. The crude product was chromatographed on silica packed with ethyl acetate/hexanes (1:1), eluting with a gradient of ethyl acetate/hexanes (1:1), ethyl acetate, 4% MeOH in ethyl acetate. Compound 22 was obtained as white solid (50% yield). MS (APCI+) m/z 1363 (M+1) detected.

### Example 20

### Synthesis of Compound 23

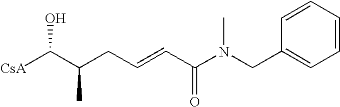

Prepared according to Procedure B, Step 2 from compound 15 (Example 13) and benzylmethylamine. The crude product was chromatographed on silica packed with ethyl acetate/hexanes (1:1), eluting with a gradient of ethyl acetate:hexanes (1:1), ethyl acetate, 4% MeOH in ethyl acetate. Compound 23 was obtained in 99% yield. MS (APCI−) m/z 1334 (M−1) detected.

### Example 21

### Synthesis of Compound 24

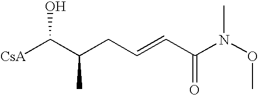

Prepared according to Procedure B, Step 2 from compound 15 (Example 13) and O,N-dimethylhydroxylamine hydrochloride. DMA was added to the mixture for solubility and the reaction was heated to 70° C. for 48 hours. The crude product was chromatographed on silica packed with ethyl acetate/hexanes (1:1), eluting with a gradient of ethyl acetate/hexanes (1:1), ethyl acetate, 4% MeOH in ethyl acetate. Compound 24 was obtained in 35% yield. MS (APCI+) m/z 1275 (M+1) detected.

### Example 22

### Procedure C: Synthesis of Compound 28

The reaction scheme for the synthesis of compound 28 according to procedure C is shown in FIG. 3.

Step 1: Synthesis of compound 25: Prepared according to Procedure A, Step 1 from cyclosporin A and 4-acetoxystyrene. The crude product was chromatographed on silica eluting with a gradient of dichloromethane, dichloromethane/MeOH (40:1), dichloromethane/MeOH (20:1), to afford compound 25 (99% yield). MS (APCI+) m/z 1322 (M+1) detected.

Step 2: Synthesis of compound 26: A solution of compound 25 (4.82 g, 3.64 mmol) in THF: ethanol (1:1) was treated with Cs₂CO₃ (1.60 g, 4.92 mmol) at room temperature for 5 hours. The reaction mixture was filtered through Celite and concentrated under reduced pressure. The residue was chromatographed on silica eluting with a gradient of dichloromethane, 2.5% MeOH in dichloromethane, and 5% MeOH in dichloromethane to provide 4.43 g of compound 26 (95% yield). MS (ESI+) m/z 1280 (M+1) detected.

Step 3: Synthesis of compound 27: To a solution of compound 26 (0.047 g, 0.037 mmol) in ethanol (0.37 mL) was added ethyl bromoacetate (0.016 g, 0.096 mmol). The mixture was stirred at room temperature for 16 hours. The reaction mixture was chromatographed on silica eluting with a gradient of 25% ethyl acetate in hexanes, 65% ethyl acetate in hexanes, and 5% MeOH in ethyl acetate to afford 41 mg of compound 27 as a white solid (81% yield). MS (APCI+) m/z 1366 (M+1) detected.

Step 4: Synthesis of compound 28: A solution of compound 27 (0.025 g, 0.018 mmol) in THF/MeOH (3:1) was treated with 5N NaOH (5 equiv.) at room temperature for 2 hours. The mixture was quenched with 5N HCl, concentrated under reduced pressure and purified by reverse phase HPLC to afford 9.3 mg of compound 28 as a white solid (38% yield). MS (ESI+) m/z 1338 (M+1) detected.

### Example 23

### Synthesis of Compound 29

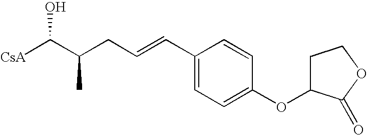

Prepared according to Procedure C, Step 3 from compound 26 (Example 22) and 3-bromodihydrofuran-2-one. The crude product was purified by reverse phase HPLC to afford 17 mg of compound 29 as a white solid (32% yield). MS (ESI+) m/z 1364 (M+1) detected.

### Example 24

### Synthesis of Compound 30

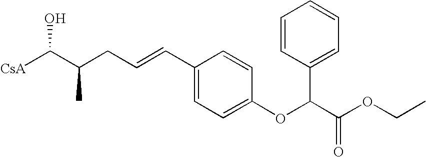

Prepared according to Procedure C, Step 3 from compound 26 (Example 22) and bromophenylacetic acid ethyl ester. The crude product was purified by reverse phase HPLC to afford compound 30 (23% yield). MS (APCI+) m/z 1442 (M+1) detected.

### Example 25

### Synthesis of Compound 31

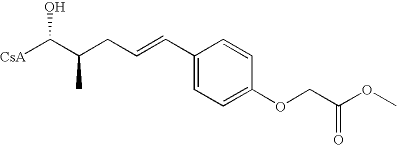

Prepared according to Procedure C, Step 3 from compound 26 (Example 22) and bromoacetic acid methyl ester. The crude product was purified by reverse phase HPLC to afford compound 31 (45% yield). MS (APCI+) m/z 1352 (M+1) detected.

### Example 26

### Synthesis of Compound 32

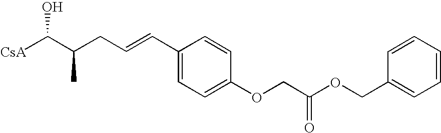

Prepared according to Procedure C, Step 3 from compound 26 (Example 22) and bromoacetic acid benzyl ester. The crude product was purified by reverse phase HPLC to afford compound 32 (15% yield). MS (APCI+) m/z 1428 (M+1) detected.

### Example 27

### Synthesis of Compound 33

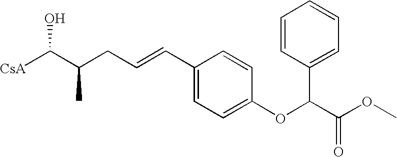

Prepared according to Procedure C, Step 3 from compound 26 (Example 22) and bromophenylacetic acid methyl ester. The crude product was purified by reverse phase HPLC to afford compound 33 (25% yield). MS (ESI+) m/z 1428 (M+1) detected.

### Example 28

### Synthesis of Compound 34

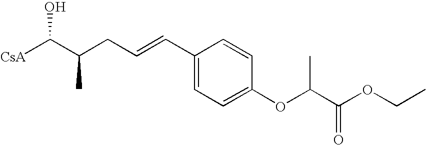

Prepared according to Procedure C, Step 3 from compound 26 (Example 22) and 2-bromopropionic acid ethyl ester. The crude product was purified by reverse phase HPLC to afford compound 34 (32% yield). MS (ESI+) m/z 1380 (M+1) detected.

### Example 29

### Synthesis of Compound 35

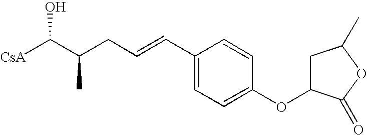

Prepared according to Procedure C, Step 3 from compound 26 (Example 22) and 3-bromo-5-methyldihydrofuran-2-one. The crude product was purified by reverse phase HPLC to afford compound 35 (46% yield). MS (APCI+) m/z 1378 (M+1) detected.

### Example 30

### Synthesis of Compound 36

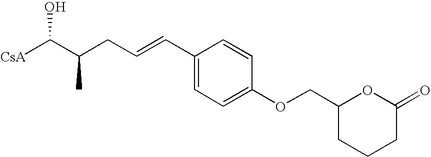

Prepared according to Procedure C, Step 3 from compound 26 (Example 22) and 6-iodomethyltetrahydropyran-2-one. The crude product was purified by reverse phase HPLC to afford compound 36 (29% yield). MS (APCI−) m/z 1391 (M−1) detected.

### Example 31

### Synthesis of Compound 37

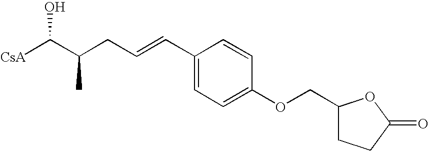

Prepared according to Procedure C, Step 3 from compound 26 (Example 22) and 5-iodomethyldihydrofuran-2-one. The crude product was purified by reverse phase HPLC to afford compound 37 (30% yield). MS (APCI−) m/z 1377 (M−1) detected.

### Example 32

### Procedure D: Synthesis of Compound 38A

The reaction scheme for the synthesis of compound 38A according to procedure D is shown in FIG. 4.

Step 1: Compound 14 was prepared as described in Example 12.

Step 2: To a solution of compound 14 (50 mg, 0.038 mmol) in acetone (0.381 mL) under N₂ atmosphere was added triethylamine (0.011 ml, 0.076 mmol) and mercaptoacetic acid ethyl ester (0.009 ml, 0.076 mmol). The reaction was heated to 50° C. for 14 hours. The reaction mixture was cooled to room temperature and concentrated in vacuo. The resulting tan solid and purified by reverse phase HPLC to afford compound 38 as a white solid (20% yield). MS (APCI+) m/z 1396.4 (M+1) detected.

### Example 33

### Synthesis of Compound 41

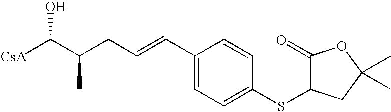

The reaction scheme for the synthesis of compound 41 according to procedure A is shown in FIG. 5.

Step 1: Synthesis of compound 39: To a solution of cyclosporin A (0.820 g, 0.682 mmol) in dichloromethane (1.7 mL) under N₂ atmosphere was added thioacetic acid S-(4-vinylphenyl)ester (1.82 g, 10.2 mmol) and Hoveyda's 2^{nd} generation catalyst (43 mg, 0.010 mmol). The resulting green solution was heated to reflux under nitrogen for 16 hours. The reaction mixture was chromatographed on silica eluting with a gradient of dichloromethane, dichloromethane/MeOH (40:1), and dichloromethane/MeOH (20:1) to afford 0.903 g of compound 39 as a gray solid (98% yield). MS (APCI+) m/z 1339 (M+1) detected.

Step 2: Synthesis of compound 40: A solution of compound 39 (0.760 g, 0.568 mmol) in 2:1 THF-MeOH (1.9 mL) was treated with 5.0 N NaOH (0.250 mL, 2.20 equivalents) at room temperature for 4 hours. The reaction was quenched with 5.0 N HCl (0.261 mL, 2.30 equivalents) and diluted with water (10 mL) and ethyl acetate (10 mL). The layers mixture was shaken and separated. The aqueous layer was extracted with an additional 10 mL of ethyl acetate. The combined organics were washed with brine solution, dried with sodium sulfate, filtered, and concentrated in vacuo. Purification on silica gel eluting with 20 to 50% acetone-hexanes provided the desired compound 40 in 55% yield. MS (APCI−) m/z 1295 (M−1) detected.

Step 3: Synthesis of compound 41: Compound 40 (0.025 g, 0.019 mmol) in dichloromethane (0.193 mL, 0.01 M) was treated sequentially with triethylamine (0.008 mL, 3.0 equivalents) and 3-bromo-5,5-dimethydihydrofuran-2-one (0.007 g, 2.0 equiv) at room temperature. After 19 hours, the reaction was concentrated in vacuo and purified by reverse phase HPLC to afford compound 41 in 11% yield. MS (APCI+) m/z 1409 (M+1) detected.

### Example 34

### Synthesis of Compound 47

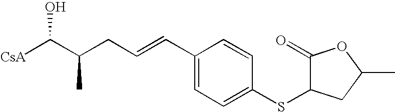

Prepared according to Procedure A, Step 1 from cyclosporin A and 5-methyl-3-(4-vinylphenylsulfanyl)-dihydrofuran-2-one The crude product was chromatographed on silica eluting with a gradient of dichloromethane, dichloromethane/MeOH (97.5:2.5) and dichloromethane/MeOH (95:5). The residue was then purified by reverse phase HPLC to afford compound 47 in 3% yield. MS (APCI+) m/z 1396 (M+1) detected.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A cyclosporin analog having the Formula (I) or a pro-drug or a pharmaceutically acceptable salt thereof: 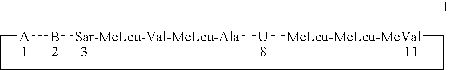
wherein residue A has the formula 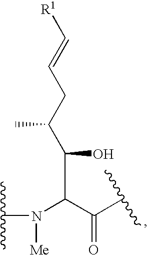
R¹ is Zₙ-heterocycloalkyl, Zₙ-heterocycloalkyl, Zₙ-S-cycloalkyl, Zₙ-S-heterocycloalkyl, ZₙS-ZₙR⁵, —C(═O)NR²R³, —(CH═CH₂)Ar, or —C(═O)O-alkyl, wherein said alkyl is substituted with phenyl, oxo or phenoxy, or 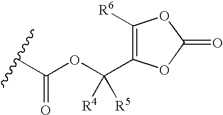
where R⁴, R⁵ and R⁶ are independently H, C₁-C₇ alkyl, alkoxyalkyl, or alkoxycarbonyl;
or R¹ is 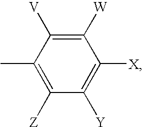
V, W, X, Y and Z are independently selected from the group consisting of H, F, Br, Cl, Zₙ-OAr, Zₙ-S-heterocycloalkyl, Zₙ-O-heterocycloalkyl, O-Zₙ-heterocycloalkyl, Zₙ-heterocycloalkyl, Zₙ-heterocycloalkyl, Zₙ-cycloalkyl, Zₙ-OAr, and Zₙ-O-alkyl, wherein at least one of V, W, X, Y or Z is other than hydrogen,
or X is 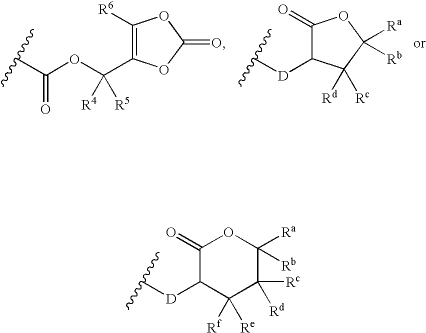
where D is O, S or C and R^{a}, R^{b}, R^{c}, R^{d}, R^{e}, and R^{f} are independently H, F, Br, Cl, alkyl, Zₙ-O-alkyl, or Zₙ-OAr,
provided that when W is F, Br, or Cl, X is other than H, and when X is F, Br, or Cl, W is other than H,
or X and Y together with the atoms to which they are attached form a substituted or unsubstituted heterocyclic ring;
R² and R³ are independently H, alkyl, Zₙ-Ar, and Zₙ-O-alkyl, wherein said alkyl and Ar may be substituted or unsubstituted;
Ar is substituted or unsubstituted aryl or heteroaryl;
Z is alkylene having from 1 to 4 carbons, or alkenylene or alkynylene each having from 2 to 4 carbons, wherein said alkylene, alkenylene, or alkynylene may be substituted or unsubstituted;
residue B is -αABu-, -Val-, -Thr-, or NVa-;
residue U is -(D)Ala-, (-D)Ser-, —[O-(2-hydroxyethyl)(D)Ser]-, —[O-acyl(D)Ser] or —[O-(2-acyloxyethyl)(D)Ser]-; and
n is 0, 1, 2, 3, or 4.

2. The cyclosporin analog of claim 1, where R¹ is (4-OPh)Ph.

3. The cyclosporin analog of claim 1, where R¹ is 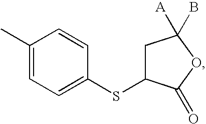
where A and B are independently H or CH₃.

4. The cyclosporin analog of claim 1, where R¹ is

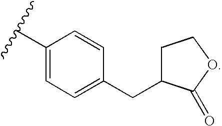

5. The cyclosporin analog of claim 1, where R¹ is

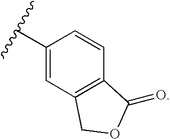

6. The cyclosporin analog of claim 1, where R¹ is

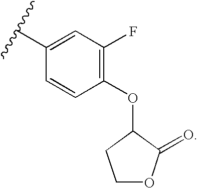

7. The cyclosporin analog of claim 1, where R¹ is

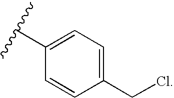

8. The cyclosporin analog of claim 1, where R¹ is —OCH₂CH₂C(═O)CH₃.

9. The cyclosporin analog of claim 1, where R¹ is

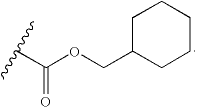

10. The cyclosporin analog of claim 1, where R¹ is

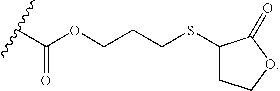

11. The cyclosporin analog of claim 1, where R¹ is

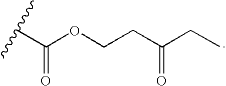

12. The cyclosporin analog of claim 1, where R¹ is

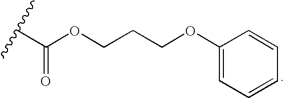

13. The cyclosporin analog of claim 1, where R¹ is

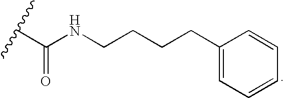

14. The cyclosporin analog of claim 1, where R¹ is

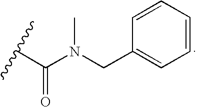

15. The cyclosporin analog of claim 1, where R¹ is

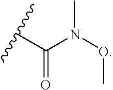

16. The cyclosporin analog of claim 1, where R¹ is

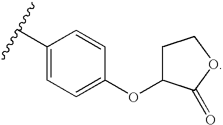

17. The cyclosporin analog of claim 1, where R¹ is

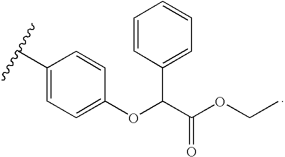

18. The cyclosporin analog of claim 1, where R¹ is

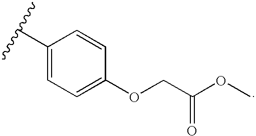

19. The cyclosporin analog of claim 1, where R¹ is

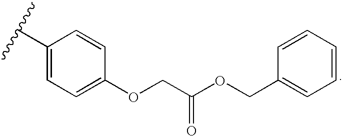

20. The cyclosporin analog of claim 1, where R¹ is

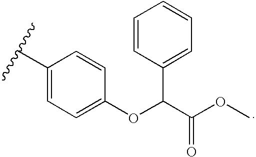

21. The cyclosporin analog of claim 1, where R¹ is

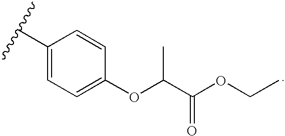

22. The cyclosporin analog of claim 1, where R¹ is

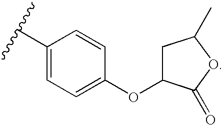

23. The cyclosporin analog of claim 1, where R¹ is

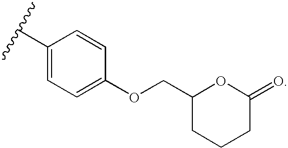

24. The cyclosporin analog of claim 1, where R¹ is

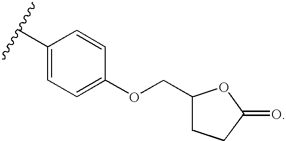

25. The cyclosporin analog of claim 1, where R¹ is

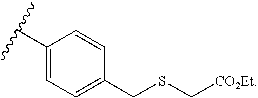

26. A method for preparing a cyclosporin analog having the Formula (I) or a pro-drug or a pharmaceutically acceptable salt thereof: 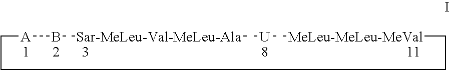
wherein residue A has the formula (II) 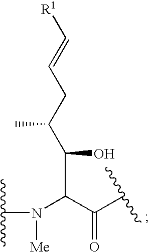
residue B is -αABu-, -Val-, -Thr-, or NVa-;
residue U is -(D)Ala-, (-D)Ser-, —[O-(2-hydroxyethyl)(D)Ser]-, —[O-acyl(D)Ser] or —[O-(2-acyloxyethyl)(D)Ser]-;
R¹ is —OR_{y} or NRₓR_{y} where Rₓ is H, OH, or a substituted or unsubstituted alky and R_{y} is H or a substituted or unsubstituted alkyl, said method comprising:
(a) treating a compound selected from the group consisting of HNRₓR_{y} or R_{y}OH with a base; and
(b) combining the product of step (a) with a compound of Formula (I) where R¹ is —CO₂Ph.

27. The method of claim 26, wherein R_{y}OH is HO(CH₂)ₘOAc.

27. The method of claim 26, wherein said base is cesium carbonate.

28. A method for preparing a cyclosporin analog having the Formula (I) or a pro-drug or a pharmaceutically acceptable salt thereof: 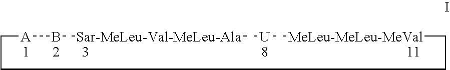
wherein residue A has the formula 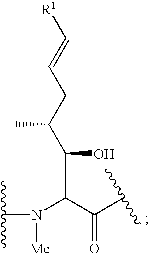
residue B is -αABu-, -Val-, -Thr-, or NVa-;
residue U is -(D)Ala-, (-D)Ser-, —[O-(2-hydroxyethyl)(D)Ser]-, —[O-acyl(D)Ser] or —[O-(2-acyloxyethyl)(D)Ser]-; and
R¹ is X(R_{z})ₘ where X is S, N, O or CN, R_{z} is H, Ar, or alkyl, m is 0, 1 or 2, and Ar is substituted or unsubstituted aryl or heteroaryl, said method comprising:
reacting a compound of formula (1) where R¹ is (2-CH₂Cl)Ph, (3-CH₂Cl)Ph, or (4-CH₂Cl)Ph with a compound having the formula HX(R_{z})ₘ in the presence of a base.

29. The method of claim 28, where X(R_{z})ₘ is (2-CH₂SCH₂CO₂Et)Ph, (3-CH₂SCH₂CO₂Et)Ph, or (4-CH₂SCH₂CO₂Et)Ph.
